(19) United States
(12) Patent Application Publication
(10) Pub. No.: US 2019/0038174 A1
(43) Pub. Date: Feb. 7, 2019

(54) DEVICE FOR DETECTING BODY FLUID BALANCE AND/OR ELECTROLYTE BALANCE

(71) Applicant: The Trendlines Group Ltd., Misgav Business Park (IL)

(72) Inventors: Avshalom SHENHAV, Haifa (IL); Arnon HADAS, Moshav Avigdor (IL); Dotan TROMER, Moshav Hosen (IL); Yosef HAZAN, Haifa (IL)

(21) Appl. No.: 16/074,796

(22) PCT Filed: Feb. 2, 2017
(86) PCT No.: PCT/IL2017/050123

§ 371 Date: Aug. 2, 2018

Related U.S. Application Data
(60) Provisional application No. 62/289,964, filed on Feb. 2, 2016.

(51) Int. Cl. A61B 5/053; A61B 5/00

(52) U.S. Cl. CPC A61B 5/0537; A61B 5/4552; A61B 5/4875; A61B 2560/0468; A61B 5/6896; A61B 2503/04; A61B 2503/10; A61B 5/682

(57) ABSTRACT

According to some embodiments there is provided a method for measuring tongue tissue impedance of an individual, comprising placing at least two electrodes in contact with tongue tissue of the individual; and measuring impedance of the tongue tissue. Some embodiments relate to a device configured for measuring tongue tissue impedance. In some embodiments, a fluid balance and/or electrolyte balance are assessed according to the measured impedance.

### RELATED APPLICATION

This application claims the benefit of priority under 35 USC § 119(e) of U.S. Provisional Patent Application No. 62/289,964 filed Feb. 2, 2016, the contents of which are incorporated herein by reference in their entirety.

### FIELD AND BACKGROUND OF THE INVENTION

The present invention, in some embodiments thereof, relates to detection of hydration status of a living body, and, more particularly, but not exclusively, to detection of fluid imbalance and/or electrolyte imbalance in the body, based on tongue tissue bioimpedance measurements.

US Patent Publication number US20140249384 A1 refers to several different bioimpedance approaches to determine a fluid status and/or dry weight, including:

“The resistance-reactance graph method (see, e.g. Piccoli et al., “A new method for monitoring body fluid variation by bioimpedance analysis: the RXc graph”, Kidney Int., 1994, 46:534-539, the disclosure of which is entirely incorporated by reference) uses whole body single frequency bioimpedance at 50 kHz for assessment of fluid status and nutritional status from height-adjusted resistance and reactance. The resulting resistance-reactance vector is set in relation to a distribution range in a normovolemic population. The difficulty of this method is that it does not provide absolute values of the fluid status—patients can only be compared to percentiles of a normal population . . . .”

“ . . . The newest and more sophisticated technique is a whole body bioimpedance spectroscopy with a physiological tissue model: wECV and wTBW are measured by whole body bioimpedance spectroscopy and additionally the fluid status and body composition are calculated. This is achieved by setting the measured patient in relation to a subject with a normal fluid status and the same body composition. Thus it relates back to the normohydrated properties of tissue. This physiologic tissue model is described in “A whole-body model to distinguish excess fluid from the hydration of major body tissues”, Chamney P. W., Wabel P., Moissl U. M. et al., Am. J. Clin. Nutr., 2007, January, 85(1):80-9, the disclosure of which is entirely incorporated by reference. This method allows the patient specific prediction of the normal fluid status and the normal fluid status weight—the weight, the patient would have with a working kidney. However the accuracy of this method can be influenced by degrees of fluid overload . . . .”

U.S. Pat. No. 5,449,000 A titled “System for body impedance data acquisition utilizing segmental impedance & multiple frequency impedance” discloses: “The unique system of the present invention provides an accurate valid measurement of human body composition consisting of fat tissue, lean tissue and body water. The inventive methodology provides a procedure for quantitative measurement of the conductive potential of the body, which is based on the lean tissue content of the body, in a convenient and reliable manner. In more detail, the quantitative measurement in accordance with the present invention is referred to as a “bio-impedance signal.” This electrical signal, in ohms, is derived from a means for measuring body impedance component of the system. The resultant signal (three digit number, between 1 and 1000 ohms) is then entered into a modifying means component to accurately predict the body composition of the tested individual.

The unique modifying component, in one embodied form, comprises prediction formulas derived from biological data inputs including: a patient's height, weight, age, and sex to determine a “population prediction variable.” Thus, the unique modifying component of the inventive system interprets bio-impedance readings as “population specific”, i.e., specific impedance values are exhibited by various pre-defined populations of individuals. This specificity is related to morph-type, leanness, body water and age.”

### SUMMARY OF THE INVENTION

According to an aspect of some embodiments of the invention, there is provided a method for measuring tongue tissue impedance of an individual, comprising placing at least two electrodes in contact with tongue tissue of the individual; and measuring impedance of the tongue tissue.

In some embodiments, a fluid-electrolyte balance is assessed according to results of the measuring.

In some embodiments, placing comprises placing the electrodes on the superior muscles of the tongue, avoiding the septum.

In some embodiments, measuring comprises stimulating the tongue tissue using the electrodes and recording a response of the tissue to the stimulation. 6. The method according to claim 5, wherein parameters of the stimulation comprise an excitation voltage between 0.1-2V.

In some embodiments, parameters of the stimulation comprise a frequency between 1 KHz-1 MHz.

In some embodiments, the method further comprises comparing impedance results of the measuring to one or more previously measured personal reference values.

In some embodiments, the method further comprises comparing impedance results of the measuring to a general reference collected from a plurality of tested individuals.

In some embodiments, the method further comprises comparing impedance results of the measuring to an impedance scale, the scale defining upper and lower limits of impedance values associated with a certain condition out of a plurality of different fluid conditions and/or electrolyte conditions.

In some embodiments, placing comprises arranging the electrodes on tongue portions in which the tissue exhibits high conductance properties relative to other tongue portions.

In some embodiments, placing comprises placing at least 4 electrodes, and wherein the measuring comprises collecting impedance samples by pairing, per each time sample collected, different electrodes selected out of electrodes that are in contact with tongue tissue out of the at least four electrodes.

According to an aspect of some embodiments of the invention, there is provided a device for measuring tongue tissue impedance as an indication of one or both of body fluid balance and an electrolyte balance of an individual, comprising: at least two electrodes sized to fit within the mouth to be positioned in contact with tongue tissue of the individual, the electrodes disposed at a distance from each other; and a controller configured for activating the at least two electrodes to measure impedance of the tongue tissue as an indication of one or both of body fluid balance and an electrolyte balance of the individual.

In some embodiments, the device further comprises an element shaped and sized to fit within the mouth, the element comprising the at least two electrodes.

In some embodiments, the element is substantially flat.

In some embodiments, the element is non-flat.

In some embodiments, the element is a PCB.

In some embodiments, the element is coated by a silicon coating.

In some embodiments, the electrodes are gold plated.

In some embodiments, the device further comprises a positioning element for holding the electrodes in the mouth, the positioning element shaped and sized to be propped against one or more anatomical structures of the mouth, internal or external.

In some embodiments, the positioning element is shaped to engage the external surface of the lips.

In some embodiments, the positioning element is shaped as a retainer.

In some embodiments, the positioning element is shaped to center a PCB on which the electrodes are mounted relative to the tongue.

In some embodiments, the device further comprises a handle configured distally to the element insertable into the mouth, the handle extending externally to the mouth for holding by a user.

In some embodiments, the handle comprises at least one port for communicating with one or more external devices out of: a computer, a smartphone, a smartwatch.

In some embodiments, the handle comprises a wireless communication module.

In some embodiments, the handle comprises a memory component.

In some embodiments, the element insertable into the mouth comprises a fuse that is short-circuited at the end of use.

In some embodiments, the handle comprises a mechanism for deforming at least a portion of the element insertable into the mouth to disable its function when disconnected from the handle.

In some embodiments, the device further comprises a temperature sensor configured to be positioned in the individual's mouth.

In some embodiments, the device is incorporated in a pacifier, for use in infants, the electrodes mounted on a nipple of the pacifier.

In some embodiments, the device is incorporated in a drinking bottle, the electrodes mounted on at least one of a mouth piece and straw of the bottle.

In some embodiments, the device is incorporated in a dog toy bone.

In some embodiments, the device is incorporated in a horse bridle.

In some embodiments, the device further comprises an air blister configured to be positioned between the electrodes and a roof of the user's mouth, the air blister configured to force the electrodes towards the tongue tissue to increase contact between the electrodes and the tissue.

In some embodiments, the distance between the electrodes is at least 20 mm.

In some embodiments, the controller is configured for activating the at least two electrodes to measure impedance of the tongue tissue by applying a current at an intensity between 0.1-5 mA.

In some embodiments, the controller is programmed to activate the electrodes over a time period of 1-3 seconds to collect at least 10 impedance samples.

In some embodiments, the device is incorporated in a mouthpiece of a hydration pack.

In some embodiments, there is provided a system for assessing one or both of body fluid balance and an electrolyte balance of an individual, comprising: a device according to claim 13; a user interface; a communication module; and a memory.

In some embodiments, the user interface is configured to provide one or more of a visible indication, an audible indication, and a tactile indication to the user.

In some embodiments, the indication comprises a current measurement status.

In some embodiments, the indication comprises one or both of a fluid balance and an electrolyte balance of the user.

In some embodiments, the indication comprises an operational alert.

In some embodiments, the alert concerns misplacing of the electrodes.

In some embodiments, the communication module is configured to provide wired or wireless communication with one or more external devices.

In some embodiments, the communication module is configured to communicate with a hospital system.

In some embodiments, for impedance values lower than 1040 ohm the system is configured to provide a dehydration alert and for impedance values higher than 1350 ohm the system is configured to provide a hyponatremia alert.

In some embodiments, the system is configured to monitor tongue impedance levels of a user and to generate a fluid consumption recommendation accordingly.

In some embodiments, the system is configured to generate a fluid and/or food consumption recommendation for a user based on a correlation between previously acquired measurements and personal physical performance levels of the user.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are a flowchart of a general method for assessing body fluid balance and/or electrolyte balance by measuring tongue tissue impedance (1A); and an exemplary graph presenting a relationship between tongue tissue impedance and body electrolyte concentration, according to some embodiments of the invention;

FIG. 2 is a flowchart of method for providing an indication related to a body fluid balance and/or an electrolyte balance by measuring tongue tissue impedance, according to some embodiments of the invention;

FIGS. 3A-D illustrate an exemplary device for measuring tongue tissue impedance (3A,C,D), according to some embodiments of the invention, and an enlarged view of the tongue (3B);

FIGS. 4A-C illustrate exemplary electrode configurations of a device for measuring tongue tissue impedance (4A-B), according to some embodiments of the invention; and an exemplary table showing electrode pairing (using the electrode configuration of FIG. 4B), for the purpose of impedance calculation, according to some embodiments of the invention;

FIG. 4D is a flowchart of a method of collecting impedance samples from the tongue, according to some embodiments of the invention;

FIG. 5 is a block diagram of a system for measuring tongue tissue impedance for providing an indication related to fluid balance and/or electrolyte balance in the body, according to some embodiments of the invention;

FIGS. 6A-C illustrate a device for measuring tongue tissue impedance in infants for providing an indication related to fluid balance and/or electrolyte balance in the body, according to some embodiments of the invention;

FIGS. 7A-B illustrate a device for measuring tongue tissue impedance for providing an indication related to a fluid balance and/or electrolyte balance in the body, the device incorporated in a drinking bottle, according to some embodiments of the invention;

FIGS. 8A-B illustrate devices for measuring tongue tissue impedance in non-human subjects, for example incorporated in a horse's bridle (FIG. 8A), or in a dog toy bone (FIG. 8B), according to some embodiments of the invention;

FIG. 9 is a graph showing exemplary tongue tissue impedance values at various physiological states (eating, drinking, running), according to some embodiments of the invention;

FIG. 10 illustrates a device for measuring tongue impedance comprising an air blister coupled to an electrode-comprising element, according to some embodiments of the invention;

FIGS. 11A-G are examples of various electrode arrangements on the tongue, according to some embodiments of the invention;

FIG. 12 presents results of tongue impedance measurements performed over time for a plurality of subjects, in accordance with some embodiments;

FIGS. 13A-E present results of tongue impedance measurements performed for a plurality of subjects before, during and following exercise (running), in accordance with some embodiments;

FIG. 14 presents results of an in vitro experiment in which impedance was measured in a physiological solution at various sodium concentrations, in accordance with some embodiments;

FIG. 15 presents results of a comparison between tongue impedance levels and urine osmolality, in accordance with some embodiments; and

FIGS. 16A-B are side view and top view images of a device for measuring tongue tissue impedance, in accordance with some embodiments.

### DESCRIPTION OF SPECIFIC EMBODIMENTS OF THE INVENTION

The present invention, in some embodiments thereof, relates to detection of fluid balance in a living body, and, more particularly, but not exclusively, to detection of fluid imbalance and/or electrolyte imbalance in the body, based on tongue tissue bioimpedance measurements.

An aspect of some embodiments relates to measuring tongue tissue bioimpedance. In some embodiments, tongue tissue impedance is measured as an indication of a body hydration balance. A potential advantage of measuring tongue tissue impedance as an indication of whole body fluid imbalance may include improved conductivity relative to, for example, impedance measurement on the epidermis.

In some embodiments, the measurement comprises placing electrodes on the tongue of a tested individual. In some embodiments, electrode positioning and/or electrode size are selected to increase the signal to noise ratio. In some embodiments, electrode positioning is selected in accordance with a conductivity level of the tissue, for example by placing the electrodes on the superior muscles of the tongue while avoiding a septum of the tongue, where tissue conductivity is significantly lower. Optionally, the electrodes are aligned symmetrically with respect to the septum. In an example, two electrodes are used, positioned on opposing sides of the tongue. In some embodiments, electrode positioning is selected so as to reduce an effect of tongue movement on the measurement.

Some embodiments relate to a device configured for tongue impedance measurement. In some embodiments, the device comprises at least two electrodes that are placed in contact with the user's tongue. In some embodiments, at least a portion of the device that is configured to be inserted into the mouth is shaped to match oral geometry. Optionally, the portion that is insertable into the mouth is flat, for example being in the form of a PCB comprising the electrodes. Alternatively, the portion that is insertable into the mouth is non-flat, for example comprising the shape of a pacifier nipple. In some embodiments, a structure of the device is designed to be propped against and/or otherwise engage one or more anatomical structures in the mouth, such as the teeth, to position the electrodes at a selected anatomical position with respect to the tongue and/or to reduce movement of the device within and/or out of the user's mouth.

In some embodiments, the anatomical position of the electrodes is selected so that the electrodes are aligned with respect to a long axis of the tongue. Additionally or alternatively, the anatomical position of the electrodes is selected to avoid tissue having lower conductivity properties, such as the tongue septum. Additionally or alternatively, the anatomical position of the electrodes is selected so that when the tongue is pushed upwards by the user, contact between the electrodes and the tongue is ensured. In some embodiments, the device comprises one or more sensors configured to detect if contact has been formed between the electrodes and the tongue. Optionally, the sensors are configured to detect the attachment force. In an example, a flex sensor is placed on an element that comprises the electrodes for indicating bending of the element which may cause the electrodes to disengage the tongue.

In some embodiments, at least a portion of the device such as the portion configured to be inserted into the mouth is disposable. Optionally, the device comprises one or more reusable portions. In an example, an element which comprises the electrodes such as a PCB is removably attached to a handle such that the element can be disconnected from the handle after use and disposed of. Optionally, a new electrode-comprising element is then attached to the handle. In some embodiments, the device is configured such that after a single use and/or after a selected number of uses and/or after a predefined time period a mechanical and/or electrical change is performed in the electrode-comprising element to disable it. Such change may include, for example, a mechanical deformation of the PCB (for example when disconnected from the handle), a short-circuited fuse, and/or other changes suitable to limit functionality of the device.

Some embodiments relate to a system for tongue impedance measurement. In some embodiments, the system comprises a controller configured to activate electrodes of the mouth piece to stimulate the tongue tissue and record the tissue response to the stimulation. In some embodiments, the system comprises a memory, for example for storing current measurement results and/or a user's history. In some embodiments, the system comprises a user interface, configured to provide a visible and/or audible and/or tactile indication to the user. In an example, the user interface comprises an LCD display, configured for example on a handle of the measuring device, for presenting a current measurement status and/or results. In another example, the user interface comprises a LED indication. In some embodiments, the system comprises a communication module, configured for wired and/or wireless communication with additional devices such a computer and/or a smart phone and/or other devices suitable for storing and/or displaying data to a user. Optionally, an associated smartphone application is configured to receive and present measured data to the user, analyze previous data, predict a future condition (for example using previously measured data of the patient) and/or provide other data to the user. In some embodiments, the system is activated via the smartphone application, for example measurements are carried out according to a preset schedule or program and/or according to user input.

In some embodiments, the system is configured to monitor the hydration state of the user. Optionally, the system warns the user upon reaching or being close to a state of dehydration and/or a state of hyponatremia. In some embodiments, the system reminds the user to perform measurement, for example according to predefined timing. Optionally, the system recommends the user an amount of fluid that should be consumed or instead avoided for improving the user's hydration status. In an example, measurements are acquired by the system early in the morning (before the user consumes any fluids or foods) and an indication of fluid amounts that should be consumed by the user over a certain time period is generated by the system.

In some embodiments, the system is configured to characterize a user based on recorded measurements. Optionally, the system advises and/or reminds the user to drink or eat based on a current measurement and/or based on previously acquired data of the user. In some embodiments, parameters of recorded data such as a variance of the measured results (e.g. of measurements performed several times over a day) and/or other parameters are analyzed. Optionally, according to the analysis, the system generates an indication of fluid amounts and/or timing of fluid or food consumption that are recommended to the user, and optionally reminds the user (e.g. via the cell phone application) to act accordingly. In some embodiments, the system analyzes impedance measurements obtained during physical activity (e.g. running or other forms of exercise). In some cases, a correlation between the impedance measurements and the performance level may exist. Optionally, based on the correlation, the system predicts a hydration condition and advises the user regarding fluid and food consumption for optimizing performance levels and/or for avoiding dehydration or hyponatremia.

In some embodiments, devices and/or systems for example as described herein may be used for assessing whether dehydration is the cause (or one of the causes) for a condition an individual is suffering from, such as fainting. In an example, impedance values are obtained from a subject that fainted to determine whether dehydration is one of the reasons that caused the fainting. Optionally, treatment is decided on according to the measured levels, for example whether to provide the user with intravenous infusion.

Some embodiments relate to control of signal to noise ratio in tongue tissue impedance measurements. In some embodiments, parameters such as electrode size, electrode positioning, electrode spacing, a manner of coupling the electrode and the tissue are selected to reduce noise. In some embodiments, measurement parameters such as a sampling rate, excitation power, excitation frequency, waveform and/or other set-up parameters are selected to reduce noise. In some embodiments, noise reduction algorithms are applied during analysis of the recorded data.

In some embodiments, impedance samples are collected by pairing, per each time sample collected, two different electrodes out of the total number of electrodes. Some potential advantages of pairing up different electrodes per each sample collected (as opposed to, for example, measuring impedance between fixed electrode pairs) may include effectively increasing the tissue surface area contacted by the electrodes, without increasing the actual number of electrodes placed on the tongue; facilitating detection of loss of contact between the electrodes and the mouth; facilitating detection of false values, and/or other advantages.

In some embodiments, impedance samples are collected by simultaneously pairing several electrode pairs and driving each electrode pair with a different frequency. A potential advantage of using different frequencies for different electrode pairs may include facilitating the impedance calculation since the electrode pairs can be easily separated, even though the electrodes were activated simultaneously.

Various embodiments of a device and/or system for example as described hereinabove may be provided, for meeting different needs and/or populations. For example, a device intended for hospital use may communicate with the hospital system. In some embodiments, the device comprises a barcode reader, which can be used, for example, to automatically insert into the device user parameters such as height, weight, medical condition, and/or other parameters taken into consideration when assessing the hydration status of the patient. In some embodiments, the user's barcode is scanned (e.g. from a bracelet worn by the user when hospitalized) before and/or during impedance measurement and the results are automatically uploaded and recorded in the user's medical record.

In another example, a device for measuring tongue impedance in infants may be incorporated in a pacifier. In another example, a device for use by athletes may be incorporated in a mouthpiece or straw of a drinking bottle. In some embodiments the device is adapted for use in non-human subjects, for example incorporated in a dog chew bone or in a horse's bridle.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As used herein, the term “proximal” may include a direction extending towards the tested individual, for example a direction extending internally into the mouth; the term “distal” may include an opposite direction, extending away from the individual and externally to the mouth.

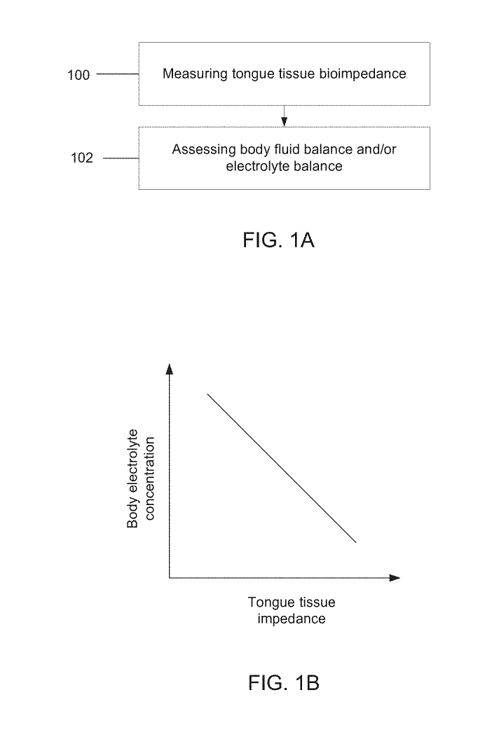

Referring now to the drawings, FIG. 1A is a flowchart of a general method for assessing a body fluid and/or electrolyte balance, according to some embodiments of the invention.

In some embodiments, the method comprises measuring impedance of tongue tissue (100). In some embodiments, a plurality of electrodes is placed in the mouth in contact with the tongue, and are activated to stimulate the tissue. In some embodiments, a response of the tissue to the stimulation is recorded and further analyzed to provide an indication related to a body fluid balance and/or electrolyte balance (102). Optionally, the stimulation comprises an electrical current at an intensity low enough such that it is not sensed by the user and does not cause movement of the tongue muscles. Optionally, the current intensity is between 0.1-5 mA, such as 0.3 mA, 4 mA, 2 mA, 4.9 mA, or intermediate, higher or lower intensities.

In some embodiments, an electrolyte balance (or imbalance) in the tested individual is assessed according to the measurement results. In an example, a state of dehydration is detected. In another example, a state of hyponatremia is detected. In some embodiments, a state of over-hydration is detected. Optionally, based on the measurement results, a state of under-hydration, normal hydration and/or over-hydration is assessed.

In some embodiments, impedance measurement of tongue tissue provides an indication of a whole body fluid balance and/or whole body electrolyte balance. Some potential advantages of measuring tongue tissue impedance as opposed to, for example, impedance measurement performed externally to the skin and involving other organs (such as arms, feet) may include a higher conductance of the tongue tissue relative to skin for example; a smaller, localized measuring site; a reduced dampening effect (as compared to the dampening effect of skin, for example); relatively homogenous tissue properties that may contribute to the reliability of the test; substantially no hard tissue; and a relatively quick response of the tongue tissue to fluid changes in the body. A fluid balance in the tongue may be equivalent to a whole body fluid balance, as blood circulates through the tongue and tongue tissue is generally affected by fluid/electrolyte balances in the blood as other body portions are. A correlation has been shown between tongue impedance levels and body water levels.

FIG. 1B is a schematic graph showing a relationship between tongue tissue impedance and body electrolyte concentration, according to some embodiments of the invention.

In some embodiments, tongue tissue impedance measurement for example as described herein is performed to assess a body electrolyte concentration. Electrolyte imbalance in a body may be caused by various factors, such as loss of body fluid, inadequate diet, malabsorption of nutrients, medication, hormonal disorders, kidney disease, heart disease and/or other factors. In some cases, an invert linear relationship exists between the tissue impedance and the body electrolyte concentration-relatively low impedance values as measured in response to tongue tissue stimulation are associated with a high body electrolyte concentration, and vice versa.

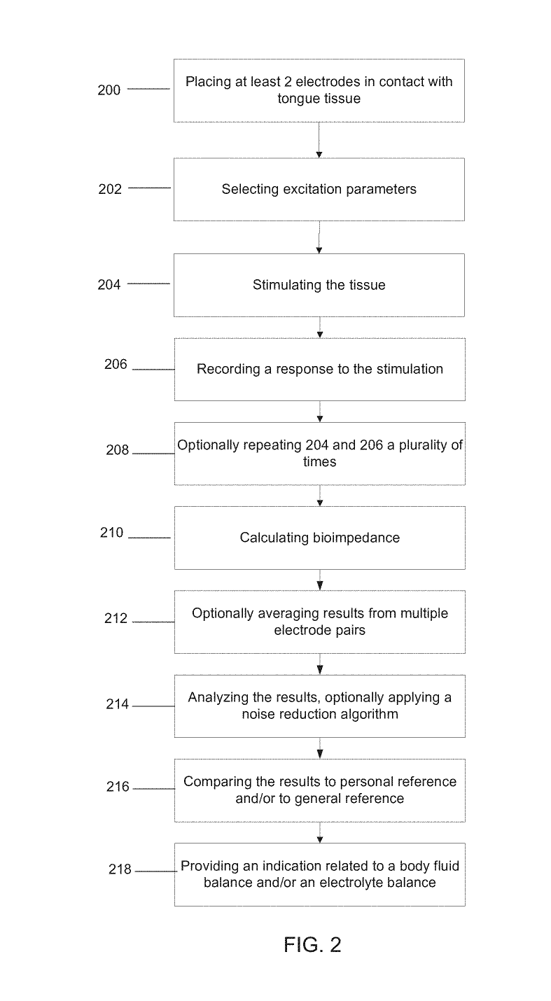

FIG. 2 is a flowchart of method for providing an indication related to a body fluid balance and/or an electrolyte balance by measuring tongue tissue impedance, according to some embodiments of the invention.

In some embodiments, two or more electrodes are positioned in the mouth, in contact with tongue tissue (200). Optionally, the electrodes are positioned on the superior longitudinal muscles of the tongue. In some embodiments, the electrodes are positioned across the septum of the tongue. In some embodiments, the electrode positioning is selected to cover a certain surface area of the tongue. In some embodiments, the electrode positioning is selected to reduce signal variability and/or noise. In some embodiments, the electrode positioning is selected so as to be least affected by tongue movement. Alternatively, tongue movement such as upwards lifting of the tongue by the user forces the electrodes in contact with the tissue. Optionally, measurement noise resulting from tongue movement is filtered during calculation, for example by selecting only a few electrode pairs (e.g. 1, 2, 4, 6 pairs) out of a plurality of electrode pairs used (e.g. 2, 3, 4, 5, 8, 12 pairs).

In some embodiments, an attachment strength between an electrode and the tissue is selected to reduce noise and/or to ensure that the electrode does not disengage the tissue. Optionally, a coupling between the electrode and tissue is achieved by the electrode being forced downwards against the tongue tissue. Optionally, the electrode is pre-shaped to contact the tissue, for example comprising a concave or convex surface which matches a curvature of the tongue tissue at a location in which the electrode is placed.

In some embodiments, a plurality of electrodes such as 2, 4, 6, 8, 10, 12, 14, 16 or a larger number of electrodes are used. Optionally, an even number of electrodes is used. Optionally, the electrodes are distributed across the septum of the tongue such that half of the electrodes are positioned left of the septum, and their paired electrodes are positioned right of the septum. Positioning the electrodes on either side of the septum may provide, in some embodiments, for detecting contact problems. In some embodiments, electrodes are positioned at multiple axial locations, along the long axis of the tongue. Optionally, the number of axial locations is selected according to the length of the tongue.

In some embodiments, excitation parameters are selected (202). Such parameters may include, for example, excitation voltage, frequency, duration, and/or other excitation parameters. In an example, the excitation voltage is between 0.1-2V, such as 0.5V, 1 V, 1.5V or intermediate, higher or lower voltage; and the frequency is between 1 KHz to 1 MHz, such as 20-70 KHz, 10-200 KHz, 150-500 KHz, 400-800 KHz, or intermediate, higher or lower frequencies.

In some embodiments, the excitation waveform is sinusoidal. Other waveforms such as squared or triangular are also contemplated.

In some embodiments, the tissue is stimulated in accordance with the selected excitation parameters (204), and the response of the tissue to the stimulation is recorded (206).

In some embodiments, excitation voltage is applied to the tissue, and an output current is recorded. Optionally, the output current is measured on a serial resistor. Additionally or alternatively, other impedance measurement methods are performed, including, for example, passing current through the electrodes and measuring the resulting voltage. Optionally, additional electrode pairs are used to measure the resulting voltage. In another example, a multi-frequency excitation is performed.

In some embodiments, a plurality of samples such as 5, 10, 15, 25, 50, 100 or intermediate, larger or smaller number of samples are collected (206). Optionally, the samples are collected over a time period of 1-3 seconds, 2-5 seconds, 1-10 seconds, 5-15 seconds, or intermediate, longer or shorter time periods.

In some embodiments, impedance of the tongue tissue is calculated. Optionally, impedance is calculated based on the applied voltage and the resulting current, by calculating the tissue's resistance and/or reactance properties. Optionally, impedance is calculated in accordance with tissue resistance and tissue inductance/capacitance properties.

In some embodiments, impedance results of multiple electrode pairs are averaged (212). Optionally, a single impedance value is arrived at, representing the tongue impedance for the current test and/or for a plurality of tests performed.

In some embodiments, the impedance results are analyzed (214). In some embodiments, absolute impedance values are used for determining the fluid balance. Additionally or alternatively, the signal phase is used for determining the fluid balance. Optionally, the signal phase is used for determining extracellular water content and/or intracellular water content and/or a ratio between them. In some embodiments, a noise-reducing algorithm is applied, for example as further described herein.

In some embodiments, the impedance results are compared to a personal reference and/or to a general reference (216), to provide the fluid and/or electrolyte balance related indication.

In some embodiments, personal reference comprises one or more previous tongue tissue impedance measurement acquired from the same individual. Additionally or alternatively, personal reference comprises impedance and/or fluid balance and/or electrolyte balance of the individual as assessed by techniques other than tongue tissue impedance measurement, for example by a blood test, a urine color and/or osmolality test, dual energy x-ray absorptiometry, and/or whole body impedance measurement.

In some embodiments, personal reference values are collected during an exercise activity, for example at time intervals such as 2 minutes, 5, minutes 10 minutes or other time intervals. Alternatively, impedance is measured continuously.

In some embodiments, the personal reference comprises an initial calibration performed at a first tongue-tissue impedance measurement of the individual. Optionally, the individual is instructed to drink a certain fluid volume prior to the measurement (optionally within a predetermined period of time), and/or to limit food consumption, in order to obtain a personally-calibrated reference of tongue-tissue impedance. Optionally, personal parameters such as weight, height, sex, and/or age are taken into consideration.

In some embodiments, the general reference comprises a database collected from a plurality of the tested subjects. Optionally, an impedance scale is defined, including upper and/or lower tongue tissue impedance limits associated with various fluid and/or electrolyte body conditions. In some cases, a current impedance measurement of the tested individual is compared to the population database to determine at least an approximated indication of the fluid and/or electrolyte balance of the individual. Optionally, personal parameters such as weight, height, sex, age are taken into consideration, and the individual is compared to a group of similar or like parameters.

In some embodiments, reference to the population database is made when only a general fluid balance indication is required and the demand for accuracy is low (e.g. very dehydrated, overhydrated, etc). Optionally, the user's measurement is compared to a population baseline averaged from a plurality of users. Reference to the population database may be advantageous in hospital-related and/or sports-related and/or military applications.

In some embodiments, reference to the personal database is made when there is a higher demand for accuracy. Optionally, even small deviations from a personal baseline of the user are detected. Reference to the personal database may be advantageous for home use applications.

In some embodiments, fluid balance and/or electrolyte balance indication is provided based on the impedance measurement (218). In an example, the indication is a general status indication (for example indicating a normal, above normal, extremely above normal, below normal, extremely below normal body fluid level and/or body electrolyte concentration. In another example, the indication provided is dehydrated/not dehydrated. Optionally, the indication is associated with the percentage of lost body water, for example under 4% body water lost would be considered tolerable, between 5-9% would be considered below normal, between 10-15% would be considered dangerously low.

In some embodiments, the indication is determined by comparing the measured value to a look-up table. Optionally, the look-up table includes measurement values and their corresponding hydration status, derived from personal and/or population-based measurements.

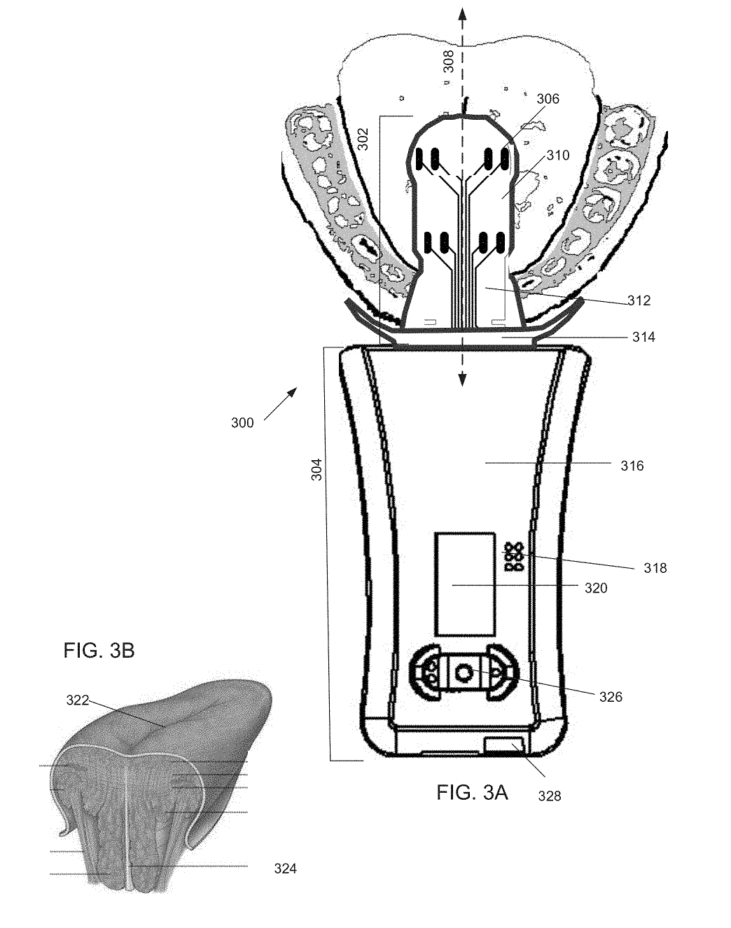

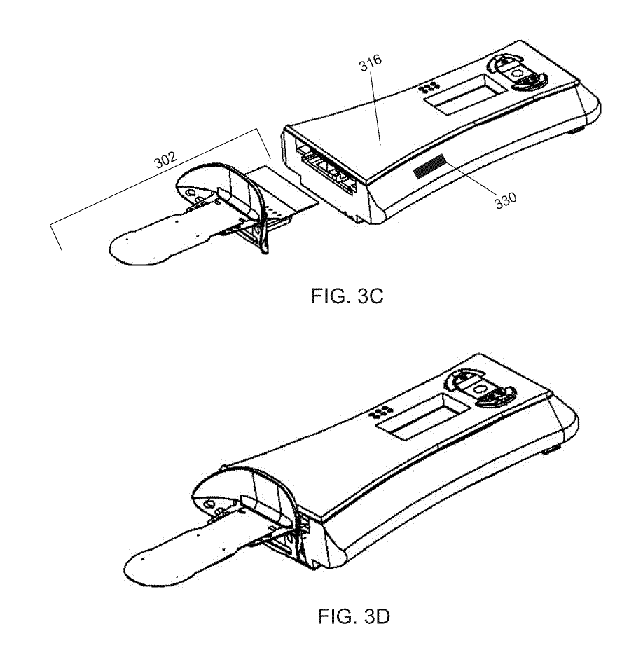

FIGS. 3A, 3C, 3D illustrate an exemplary device 300 for measuring tongue tissue impedance, and an exemplary positioning of the device in the mouth, according to some embodiments of the invention.

In some embodiments, device 300 comprises a first proximal portion 302 insertable, at least in part, into the mouth of an individual. In some embodiments, the device comprises a second portion 304 distal portion to portion 302 which is configured to be positioned, at least in part, outside the mouth.

In some embodiments, first portion 302 comprises two or more electrodes 306. In the exemplary configuration shown herein, the device comprises 4 pairs of electrodes, distributed symmetrically relative to a long axis 308 of portion 302.

In some embodiments, the electrodes are connected to an element shaped and/or sized to fit within the mouth, the element configured to form contact between the electrodes and the tissue. In some embodiments, the electrode-comprising element is flat. Alternatively, the element is non-flat, for example comprising the shape of a pacifier nipple. In some embodiments, the element is rigid. Alternatively, the element is soft. Optionally, the element is flexible.

In the example shown herein, the electrode-comprising element is a PCB 310. Optionally, the electrodes are disposed on a distally facing surface 312 of the PCB, so as to face tongue tissue when portion 302 is inserted into the mouth. In some embodiments, PCB 310 is shaped as an elongated strip, tab, and/or any other configuration shaped and sized for insertion into the mouth. In some embodiments, PCB 310 is coated with a soft material, such as silicon or polyurethane. Optionally, the coating improves conductivity. Optionally, the coating reduces damage to tongue tissue, such as irritation or abrasion of the tissue by the PCB.

In some embodiments, the electrode comprising element includes any structural arrangement in which the electrodes (and/or wiring of the electrodes) are held at a predetermined distance and/or orientation relative to each other.

In some embodiments, device 300 comprises a positioning element 314. In some embodiments, the positioning element is configured to engage one or more anatomical structures of the mouth, such as teeth, lips, jaws, and/or any other part of the mouth, internally and/or externally. Optionally, the positioning element is configured distally to the electrodes. In some embodiments, the positioning element is ergonomically shaped to engage at least a portion of the contour of the mouth. In some embodiments, the positioning element 314 is configured to position and/or to maintain a position of the electrodes in the mouth. Optionally, the positioning element is configured to center the PCB relative to the tongue. In some embodiments, the positioning element is configured to reduce tongue movement during the measurement, for example by pressing the PCB downwards onto the tongue surface. In some embodiments, positioning element 314 is shaped to engage the lips of the individual (for example in a pacifier-like manner). Additionally or alternatively, positioning element 314 is shaped engage the individual's teeth, for example being shaped as a retainer. Optionally, positioning element 314 is shaped to match a contour of the lips. Optionally, positioning element 314 is sized to fit between the teeth and the lips. In some embodiments, positioning element 314 comprises protrusions for clinging to the lips of the user.

In some embodiments, second portion 304 of device 300 comprises a handle 316, extending distally and at least partially externally to the mouth to be engaged by a user. Optionally, handle 316 is shaped for gripping by a user. In some embodiments, handle 316 comprises a user interface, for example in the form of one or more operational buttons 318 (such as an on/off button), and/or a screen 320 (such as an LCD screen) for displaying information. Optionally, screen 320 is a touch screen. Exemplary information displayed on the screen may include, for example, the calculated impedance value, a body fluid balance indication, a body electrolyte balance, remaining time of an on-going measurement, a number of samples acquired, personal parameters, history of personal results, and/or any other parameters.

In some embodiments, device 300 comprises a LED indication 326. Optionally, the LED indication is configured in a visually accessible location, such as in the handle. In some embodiments, the LED indicates a current measurement status: for example, no light means that the device is ready for the next measurement.

In some embodiments, the device comprises a port 328 for communicating with additional devices, such as a computer, smartphone, hospital equipment and/or other devices. In an example, the port comprises a USB port.

In some embodiments, the device is configured for wired communication with devices for example as described herein. Additionally or alternatively, the device is configured for wireless communication (for example via a bluetooth connection).

In some embodiments, handle 316 houses a battery (not shown herein). In some embodiments, handle 316 houses a memory component, for example a microSD card.

In some embodiments, in use, the electrodes are positioned to contact the superior longitudinal muscles 322 of the tongue (see FIG. 3B, illustrating an anatomy of the tongue). In some embodiments, the electrodes are positioned across a septum 324 of the tongue. In some embodiments, the electrodes are positioned in accordance with conduction properties of the tissue—for example, the septum is avoided since it comprises tissue exhibiting relatively low conductivity. In some embodiments, the electrodes are distributed relative to a long axis of the tongue. Optionally, at least some of the electrodes are placed at a posterior position, and at least some electrodes are placed in an anterior tongue position (i.e. closer to the lips). Optionally, side edges of the tongue and/or an anterior end portion of the tongue are avoided, since tissue in these portions may be non-uniform and/or too lean to provide for a reliable impedance measurement. In some embodiments, the electrodes are positioned only at muscle tissue locations.

In some embodiments, one or more electrodes are placed to contact the tongue from an inferior position. Optionally, in a device configured for engaging the tongue inferiorly, an element for example in the form of a fastener may be provided to couple the electrodes to the tongue and ensure contact between the electrodes and the tissue.

In some embodiments, one or more electrodes are placed on an inner side of the lips.

In some embodiments, at least one electrode is placed in a first anatomical portion of the mouth, and at least one electrode is placed on a second anatomical portion of the mouth, for example, a first electrode is positioned on the tongue and a second electrode is positioned on the inner side of the lips, a first electrode is positioned on the superior side of the tongue and a second is positioned on the inferior side of the tongue, and/or other combinations. Optionally, a noise reduction algorithm is selected according to the anatomical locations of the electrodes.

In some embodiments, mechanical means such as applying of pressure are used in order to temporarily hold the electrodes at their selected tissue location.

Further aspects of the device, according to some embodiments of the invention, are shown in the isometric views of FIGS. 3C and 3D. In some embodiments, portion 302 which is configured to be inserted at least in part into the individual's mouth is disposable, and can be disconnected from the handle and replaced. In some embodiments, handle 316 comprises a switch 330 for releasing portion 302.

In some embodiments, portion 302 is replaced if the device is transferred from one person to another. In some embodiments, portion 302 is replaced after every use. Additionally or alternatively, portion 302 is replaced periodically, for example after a selected number of uses and/or after a certain period of time, for example every week, every month, every year or intermediate, longer or shorter time periods.

In some embodiments, the device comprises one or more mechanisms for ensuring personal use and/or otherwise limited use of the device (e.g. single use). Such mechanisms include, for example, a fuse (optionally embedded in the PCB) that is short-circuited at the end of use; a mechanical deformation of the PCB during detachment from the handle that will affect the PCB function (for example by cutting out a portion and/or by punching a hole to disconnect wiring), and/or other mechanisms.

In some embodiments, the device can be used over time to enable prediction of future fluid and/or electrolyte imbalance. Optionally, the device notifies the user when detecting levels that were previously associated with fluid and/or electrolyte imbalance that leads to dehydration and/or other conditions in the specific user.

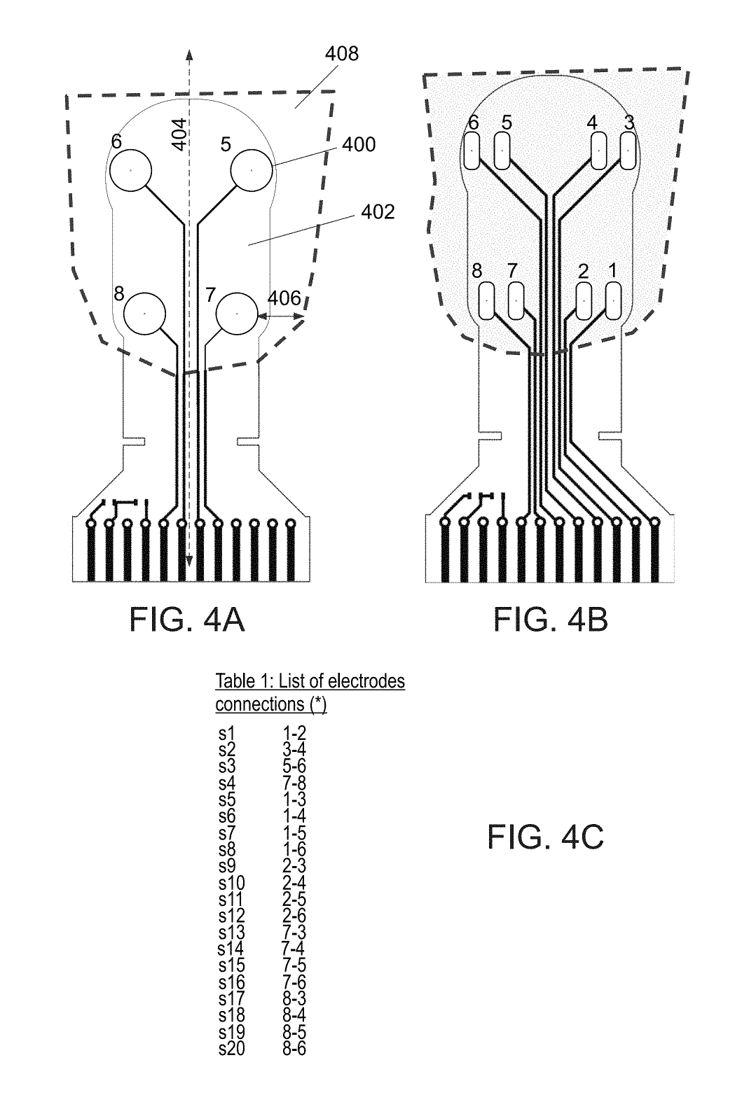

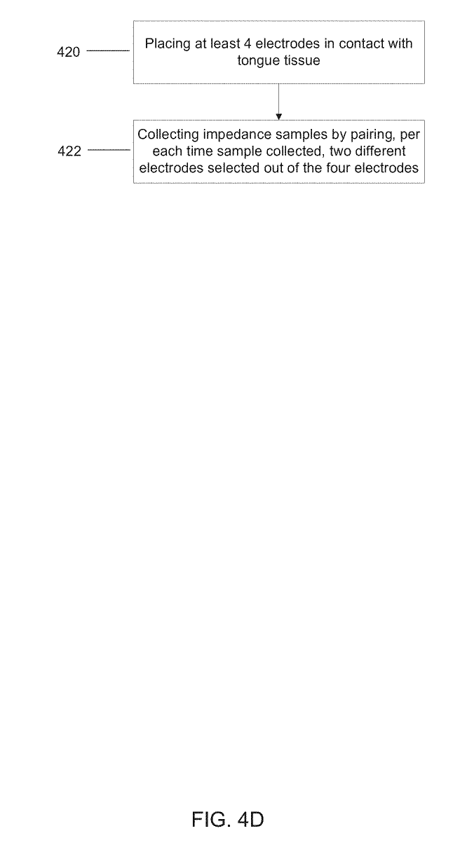

FIGS. 4A-B illustrate two exemplary electrode configurations, according to some embodiments of the invention; FIG. 4C is an exemplary table showing electrode pairing (using the configuration of FIG. 4B), for the purpose of impedance calculation, according to some embodiments of the invention.

In the exemplary configurations shown herein, FIG. 4A illustrates a configuration including 4 electrodes, and FIG. 4B illustrates a configuration including 8 electrodes. It is notes that a smaller, intermediate, or higher number of electrodes may be used.

In some embodiments, the electrodes 400 are arranged symmetrically relative to a long axis 404 of PCB 402. Alternatively, the electrodes are arranged asymmetrically. In some embodiments, the electrodes are arranged to contact tongue portions in which the tissue is relatively homogenous in its conductance properties, for example avoiding portions comprising tissue other than muscle tissue, such as tendons. In some embodiments, the electrodes are positioned a distance 406 away from the side edges of the tongue 408. In some embodiments, the electrodes are spaced apart from each other a distance of at least 20 mm, at least 10 mm, at least 5 mm, at least 30 mm, or intermediate, longer or shorter distances. Optionally, the distance is selected to provide an optimal signal to noise ratio.

In some embodiments, a number and/or arrangement and/or size of electrodes 400 on PCB 402 is selected to improve the signal to noise ratio of the impedance measurements. Noise may be affected by one or more of: electronic and digital noise; noise caused by a non-tight coupling between the electrode and tongue tissue; noise caused by misalignment of the electrode relative to the tongue; and/or other sources of noise.

In some embodiments, to reduce noise, a user waits a certain period of time (e.g. 1 minute, 5 minutes, 10 minutes, 30 minutes or intermediate, longer or shorter time periods) after drinking or eating and only then takes the measurement. In some embodiments, measurement is performed when the mouth is clear of any food or drink residue. In some embodiments, a user may wipe the tongue surface before placing the device inside the mouth.

FIG. 4D is a flowchart of a general method for collecting tongue impedance samples, according some embodiments of the invention.

In some embodiments, a total number of n electrodes, for example at least 2 electrodes, at least 4 electrodes, at least 8 electrodes, at least 12 electrodes or intermediate, higher or lower number of electrodes are placed in contact with tongue tissue (420). In some embodiments, impedance samples are collected by pairing, per each time sample collected, two different electrodes selected out of the total number of electrodes (422). For example, as shown in the table of FIG. 4C, a time-sample (see s1, s2, s3 etc in the table) is calculated from a set of 2 electrodes selected out of the total of 8 electrodes. A potential advantage of collecting data in the above described manner (i.e. pairing up different electrodes per each time sample) may include that the effective tissue area covered by the electrodes is substantially increased in size, allowing to average-out the noise, while the actual number of electrodes used is smaller than the number of electrodes that would have been required if impedance was calculated only between fixed electrode pairs, and not between combinations thereof. Another potential advantage of a method as described hereinabove in which impedance is measured and calculated between combinations of electrode pairs and not between fixed (or constant) electrode pairs may include identifying exceptional and/or false values more easily, (occurring for example as a result of an electrode being misplaced, for example being positioned over the septum). Another potential advantage may include detecting electrode misalignment and/or loss of contact with the tissue.

In some embodiments, a size of the electrode is selected in accordance with the total tissue surface area that needs to be covered. In an example, an electrode comprises a contact surface area 410 between 6 mm̂2 to 22 mm̂2, such as 10 mm̂2, 15 mm̂2, 20 mm̂2 or intermediate, larger or smaller contact surface area.

In some embodiments, a tissue contacting portion of the electrode comprises a non-corrosive material, for example gold and/or platinum.

In some embodiments, as shown in the exemplary table of FIG. 4C, electrode combinations include pairing of electrodes along a long axis of the tongue (see for example s5-s20), and/or pairing of electrodes along a horizontal axis of the tongue (see for example s1-s4).

The following describes an exemplary calculation performed to reduce noise, it is noted that other methods and/or other condition and/or other parameters may be used for performing such calculation:

assuming tissue homogeneity along a long axis of the tongue, and assuming that the samples are independent, a positioning-related noise should be reduced by √n when averaged over the n electrodes.

An average impedance result for a single time sample is: R=average (S5 . . . S20). In order to identify exceptions, each time sample is taken into consideration only if the following condition exists:

MAX(ABS((S₅ . . . S₂₀)−R))<MIN(R*th,STD(S5 . . . S20)*4)

Where “th” is a threshold parameter having a default value=0.07, defining a deviation limit of 7% from the average R. It is noted that intermediate, smaller or higher thresholds may be applied.

In some embodiments, a noise algorithm is selected and/or modified in accordance with a position of the electrodes relative to the tongue. In an example, when electrodes are positioned at an inferior position the measured signal may be noisier as compared to electrodes positioned at a superior position, and a different threshold may be used when applying the noise reduction algorithm.

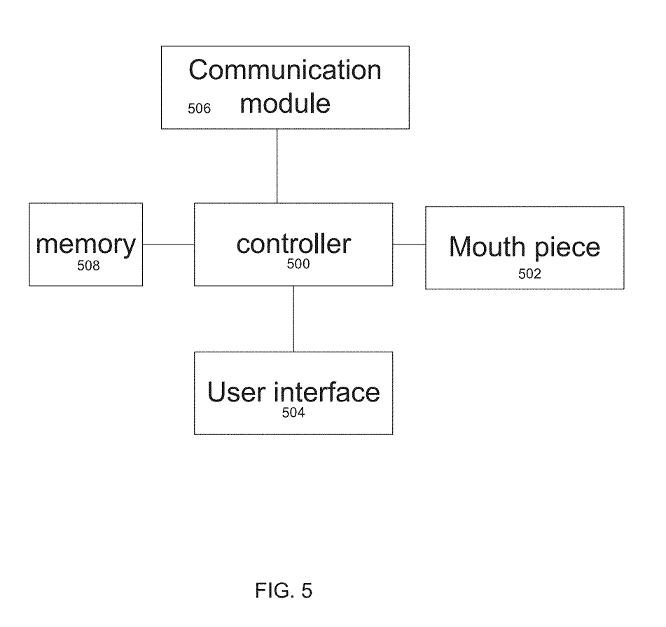

FIG. 5 is a block diagram of a system for measuring tongue tissue impedance for providing an indication related to a fluid balance and/or electrolyte balance in the body, according to some embodiments of the invention.

In some embodiments, the system comprises a controller 500, a mouth piece 502, a user interface 504, and optionally a communication module 506, and/or memory 508.

In some embodiments, mouth piece 502 comprises a plurality of electrodes positionable within the individual's mouth and configured to contact tongue tissue.

In some embodiments, controller 500 is configured to activate the electrodes of mouth piece 502 to stimulate the tissue. In some embodiments, controller 500 is configured to record and/or analyze the measurement results. Optionally, electrode activation is executed according to a predetermined protocol.

In some embodiments, electrode activation is performed via an analog electronic circuit, comprising a first channel for the excitation signal and a second channel for sampling the current in response to the stimulation. In some embodiments, the circuit comprises a multiplexer for selecting the input channels (electrode pairs).

In some embodiments, the measurement results are stored in memory 508. Optionally, memory 508 is configured to store additional data such as previous measurement results of the user, user information, activation protocols, and/or other data. In an example, the memory comprises a microSD card. Optionally, the memory is accessible to a user and can be removed from the device, for example for transferring the results to a computer, smartphone and/or other device.

In some embodiments, measurement results, a current measurement status, personal data and/or other information are conveyed to the user via user interface 504. In some embodiments, the user interface comprises a screen display, for example an LCD display. Optionally, the display is incorporated in a handle of the device. In some embodiments, the user interface comprises a visual and/or audible and/or tactile indication to the user. A visible indication may include, for example, a LED indicating a current measurement status and/or a fluid balance status; an audible indication may include, for example, a beeping sound indicating a measurement status (for example that the measurement is completed and the mouth piece can be removed from the mouth); a tactile indication may include, for example, vibration of the handle for indicating a measurement status; and/or other indications.

In some embodiments, the user interface is configured to notify a user about a current position of the mouth piece, for example alerting that the electrodes are misplaced, that the battery is running out, and/or provide other operation related notifications.

In some embodiments, communication module 506 is configured to provide wired and/or wireless communication to additional devices such as a computer, a smartphone, a smart watch, hospital equipment, and/or others.

In some embodiments, the system is configured to provide indications other than a fluid balance and/or electrolyte balance, for example a temperature indication. In some embodiments, the mouth piece comprises a temperature sensor. A temperature indication may provide an advantage when diagnosing a balance of the individual, when provided along with a fluid/electrolyte balance. Additionally or alternatively, the mouth piece comprises a PH sensor. Additionally or alternatively, the mouth piece comprises a pulse oximeter.

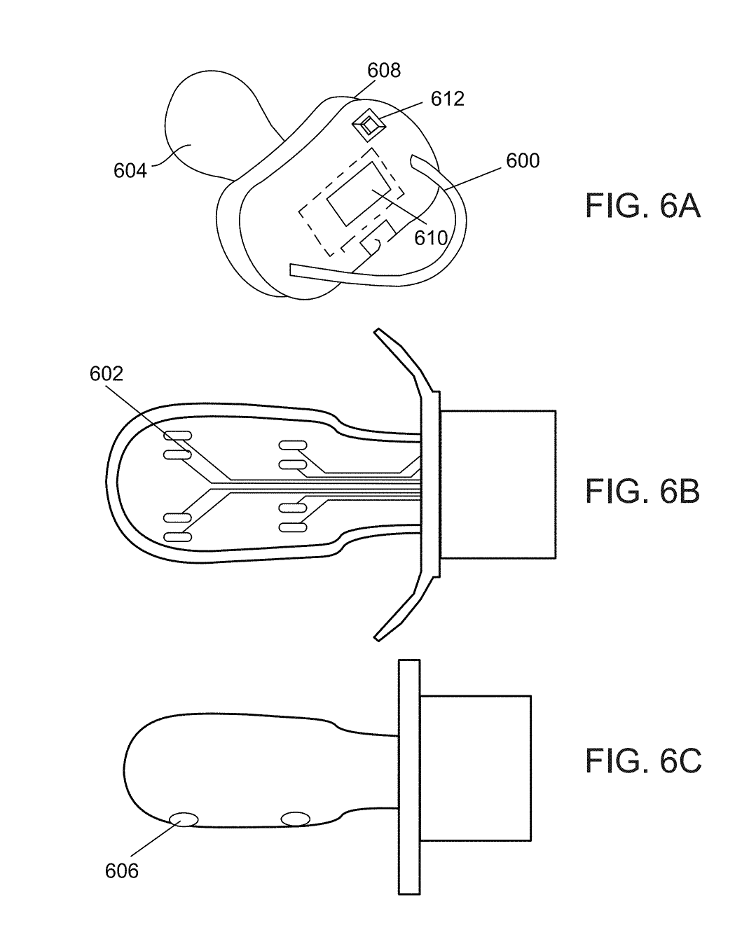

The following description of FIGS. 6A-C to 9 describes examples of tongue impedance measurement devices designed for different populations.

FIGS. 6A-C illustrate a device for measuring tongue tissue impedance in infants, according to some embodiments of the invention.

In some embodiments device 600 is shaped as a pacifier, to be used for indicating a fluid and/or electrolyte balance in small babies and children. In some embodiments, the plurality of electrodes 602 are incorporated into the nipple 604 of the pacifier. Optionally, the nipple further comprises one or more temperature sensors 606.

In some embodiments, a guard 608 which is pressed against the infant's lips comprises a screen display 610 for indicating the measurement results and/or a measurement status. In some embodiments, guard 608 further comprises a port 612 for connecting the pacifier to a different device, for example for transferring data to a computer.

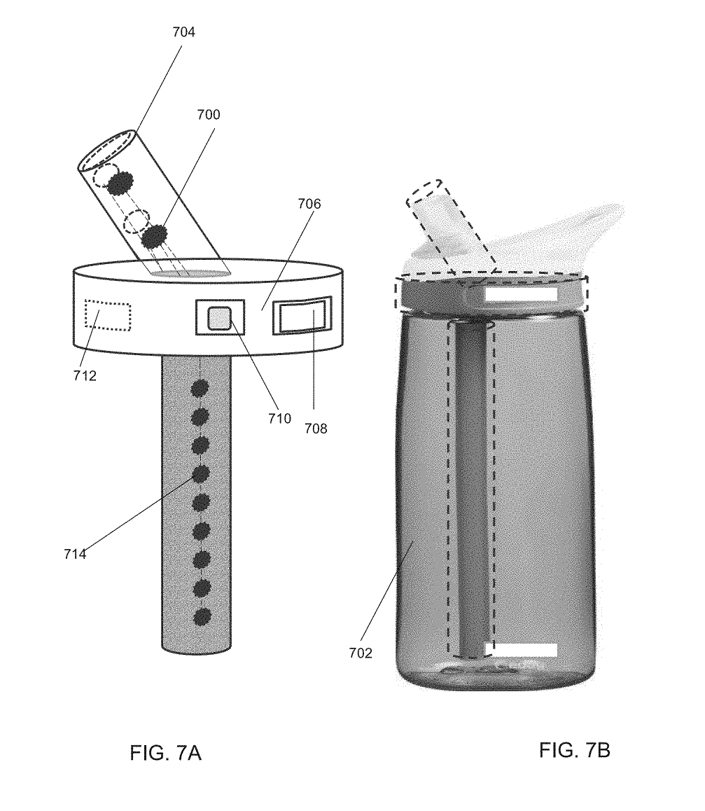

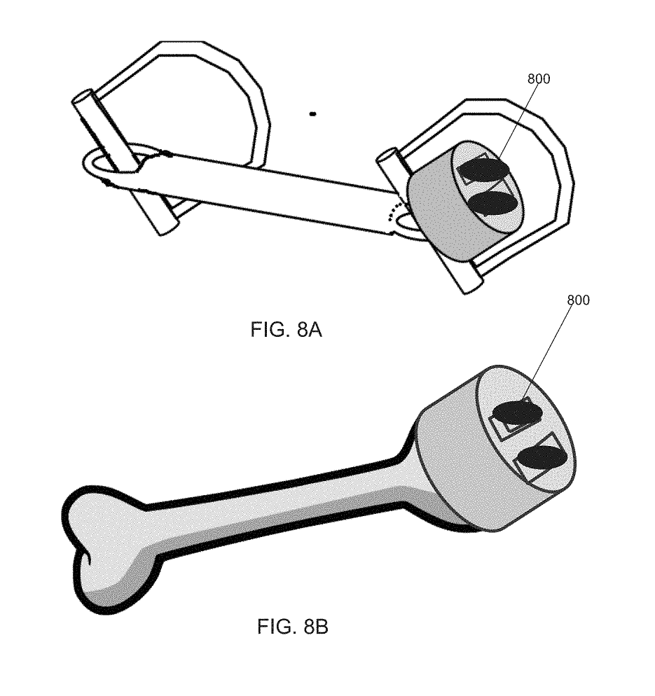

FIGS. 7A-B illustrate a device for measuring tongue tissue impedance incorporated in a drinking bottle, according to some embodiments of the invention.

In some embodiments, a plurality of electrodes 700 are incorporated in the mouth piece of a drinking bottle 702. In some embodiments, the electrodes are positioned on a straw-like element 704 which is configured to contact the tongue when inserted into the individual's mouth. In some embodiments, a cap 706 (and/or other part of the bottle) comprises a screen display 708, for indicating the measurement results and/or a measurement status. In some embodiments, cap 706 comprises one or more activation buttons 710. In some embodiments, cap 706 comprises a port 712 for connecting to additional devices.

In some embodiments, sensors 714 are configured along the length of straw 704, for detecting an amount of liquid in the bottle. Sensors 714 may include electrodes such as electrodes 700 and/or any other sensors configured for detecting presence of liquid.

A drinking bottle for example as described herein may be especially advantageous for military applications and/or athletic applications and/or any other applications in which a close follow-up on hydration status is required or advantageous.

In some embodiments, a device for measuring tongue tissue impedance for example as described hereinabove is incorporated in a mouthpiece of a hydration pack and/or any other personal portable hydration systems.

FIGS. 8A-B illustrate devices for measuring tongue tissue impedance in non-human subjects, for example incorporated in a horse's bridle (FIG. 8A), or in a dog toy bone (FIG. 8B), according to some embodiments of the invention. Optionally, electrodes 800 are placed in the animal's mouth such that they contact the animal's tongue. Optionally, a caretaker of the animal holds the device in the animal's mouth when measuring. Additionally or alternatively, supporting elements such as the bridle are used.

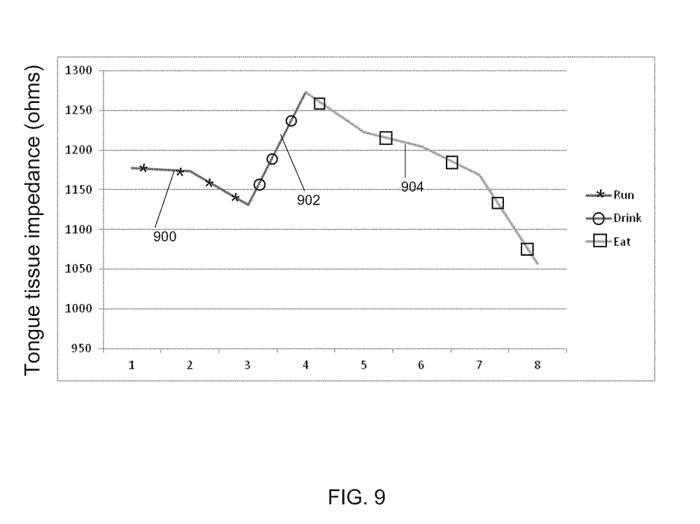

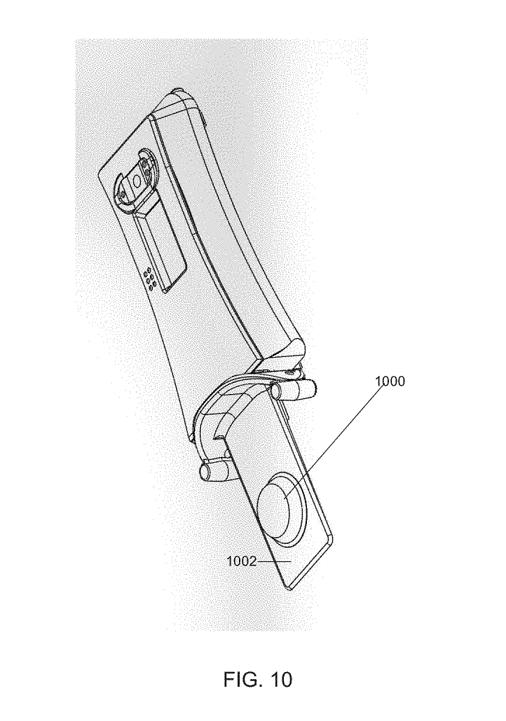

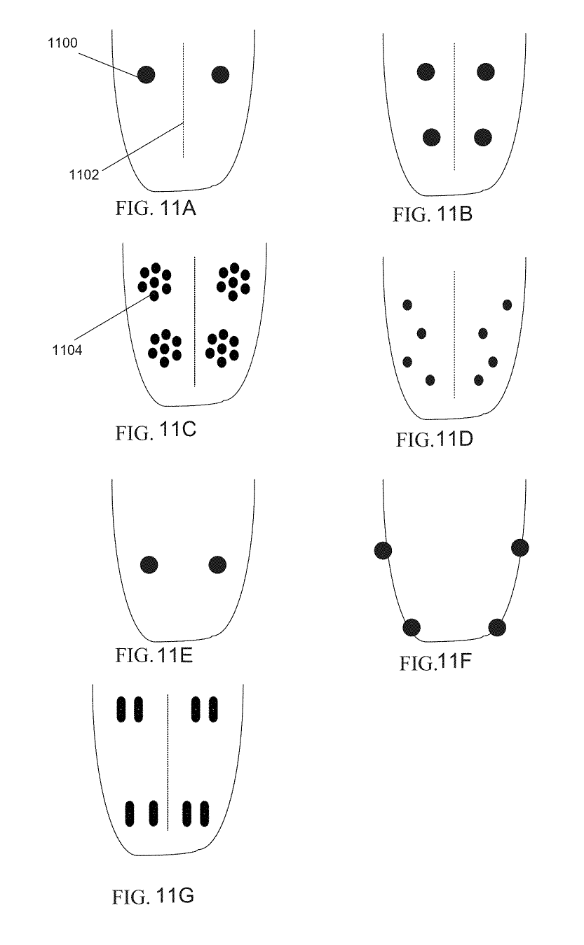

FIG. 9 is a graph showing exemplary tongue tissue impedance values at various physiological states (eating, drinking, running), according to some embodiments of the invention.

Various factors affect a body fluid and/or electrolyte balance. The graph of FIG. 9 presents a relationship between a physiological state and the tongue tissue impedance values associated with that state.

For example, a physical exercise such as running (900) may cause loss of one or both of body water and/or sodium (and/or other electrolytes). The tissue conductance may depend on the extent in which each is reduced. When drinking (902), the electrolyte concentration decreases, causing a rise in tissue impedance. When eating (904), the electrolyte concentration is increased, causing a reduction in tissue impedance.

FIG. 10 illustrates a device for measuring tongue impedance comprising an air blister 1000 coupled to an electrode-comprising element, according to some embodiments of the invention.

In some embodiments, air blister 1000 comprises an air-filled pocket configured to be positioned between the electrodes (or the electrode-comprising element 1002) and the inner walls of the mouth, for example from the roof of the mouth. Optionally, blister 1000 extends from element 1002 in an upwards direction towards the roof of the mouth. When element 1002 is inserted into the user's mouth, pressure is applied to blister 1000 (for example upon closure of the jaw over element 1002), pushing down on the electrodes of element 1002 (not shown herein) to increase contact between the electrodes and the tongue tissue.

FIGS. 11A-G are examples of various electrode arrangements on the tongue, according to some embodiments of the invention.

In some embodiments, one or more electrodes are positioned on the superior portions of the tongue. Additionally or alternatively, one or more electrodes are positioned on the inferior portions of the tongue. Additionally or alternatively, one or more electrodes are positioned on side edges and/or front edges of the tongue. Optionally, electrodes are symmetrical with respect to a long axis 1106 and/or with respect to a short axis 1108 of the tongue. Additionally or alternatively, electrodes are asymmetrical with respect to the long axis and/or with respect to the short axis of the tongue. In some embodiments, electrode pairs are positioned with equal distances between the electrodes forming the pair. Alternatively, different electrode pairs have different distances defined between the electrodes forming the pair.

In some embodiments, electrodes are arranged in groups. For example, a group may include 2 electrodes, 4 electrodes, 5 electrodes, 7 electrodes as shown herein in FIG. 11C, 10 electrodes or intermediate, larger or smaller number of electrodes.

Optionally, electrodes of a single group are connected together to produce a single signal during measurement.

FIG. 11A, two electrodes 1100 are positioned across each other and across (i.e. left and right of) the septum 1102, on the superior longitudinal muscles of the tongue, according to some embodiments. FIG. 11B illustrates a similar 4-electrode arrangement, according to some embodiments. FIG. 11C illustrates 4 sets of electrode groups 1104, according to some embodiments. FIG. 11D illustrates 8 electrodes distributed across the tongue tissue, with varying distances between opposite electrode pairs, according to some embodiments. FIG. 11E illustrates two electrodes positioned on the inferior longitudinal muscles of the tongue, according to some embodiments. FIG. 11F illustrates 4 electrodes positioned at the tongue side and/or front edges, according to some embodiments. FIG. 11G illustrates use of elongated electrodes, according to some embodiments. It is noted that the configurations shown herein are only examples of possible configurations, and that other electrode arrangements and/or combinations of the above and/or electrode shapes are contemplated as well.

A Set of Experiments Performed by the Inventors

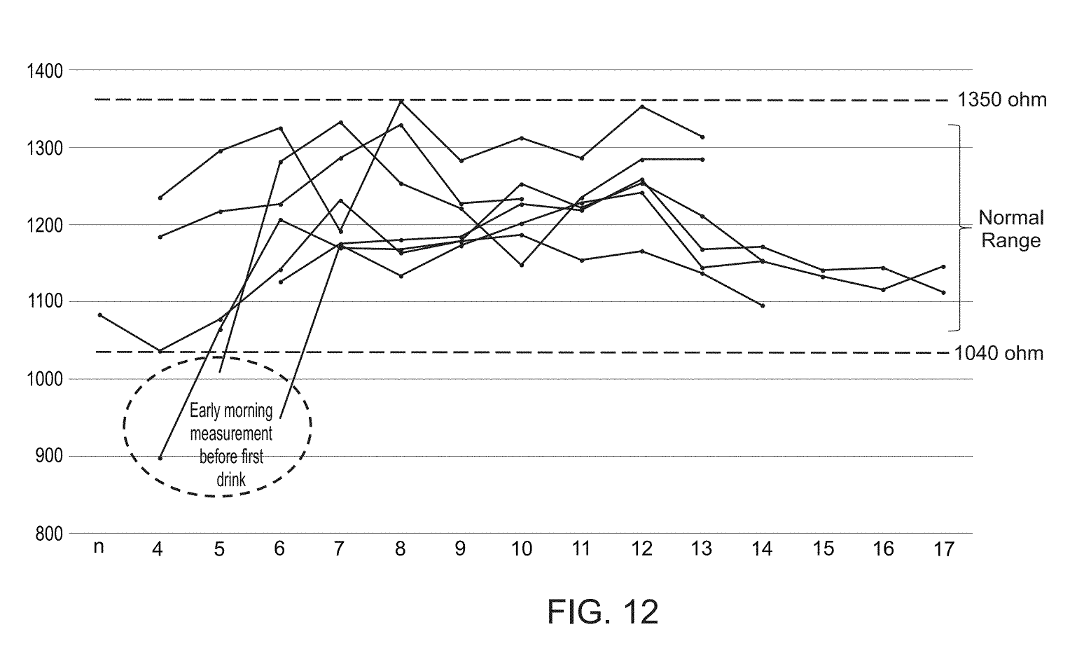

FIG. 12 presents results of tongue impedance measurements performed over time for a plurality of subjects, in accordance with some embodiments. In this experiment, tongue tissue impedance was measured in 8 subjects. For each of the subjects, measurements were acquired about 10 times over the course of approximately 14 hours. The subjects were asked to perform normal day activities and maintain their usual eating and drinking habits.

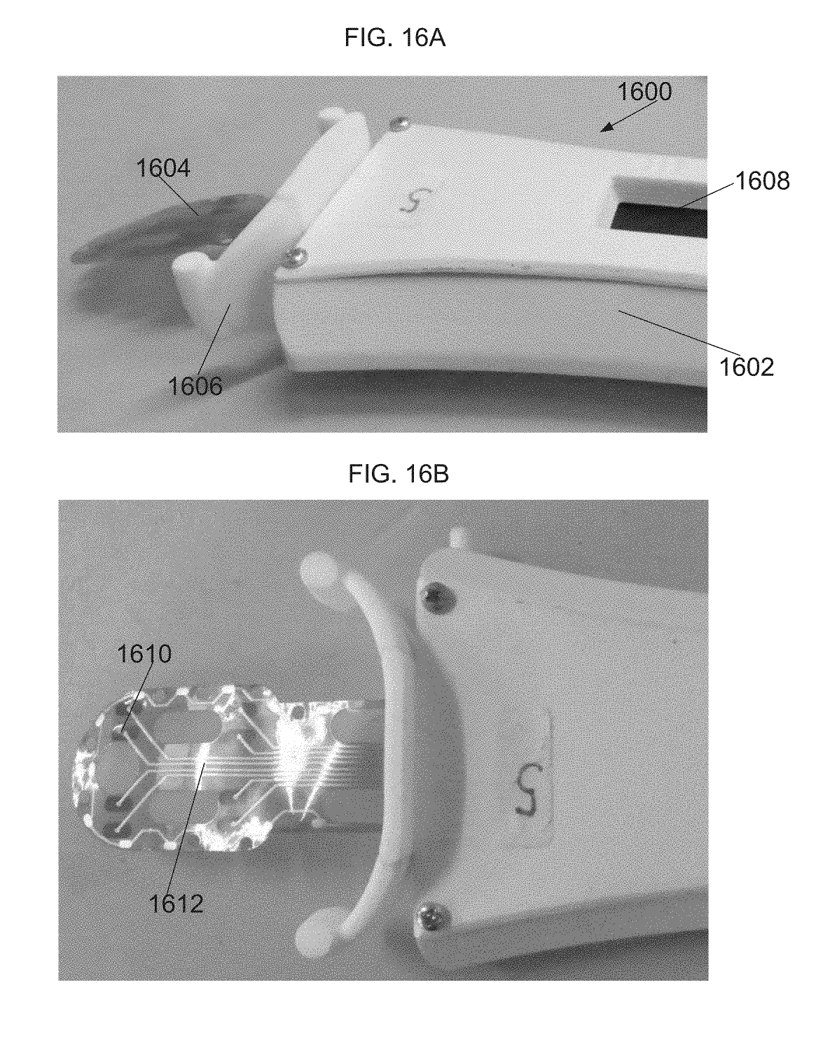

For obtaining measurements, a device for measuring tongue tissue impedance for example as shown in FIGS. 16A-B was used. The device included an 8-electrode arrangement for example as shown hereinabove in FIG. 4B. In operation, an excitation voltage of 2V was applied. Electrode pairs were scanned with a 100 ms delay between each of the pairs, in an order for example as described in FIG. 4C (according to electrode pairings S5-S20). For calculating a single impedance value for a certain time point, the electrode pair measurements were averaged. The calculation excluded the 2 lowest and 2 highest extremities, in attempt to reduce noise. The presented results suggest that most impedance values (except for early morning measurements obtained before any drinking or eating) were within the normal impedance range defined between 1040 ohm and 1350 ohm, whereas lower values indicate a state of dehydration and higher values indicate a stage of hyponatremia. In some embodiments, for example as performed in this experiment, an impedance range is defined taking into account one or more of: the fact that impedance is measured from the tongue; the electrode size; the electrode type; distance between the electrodes and/or others.

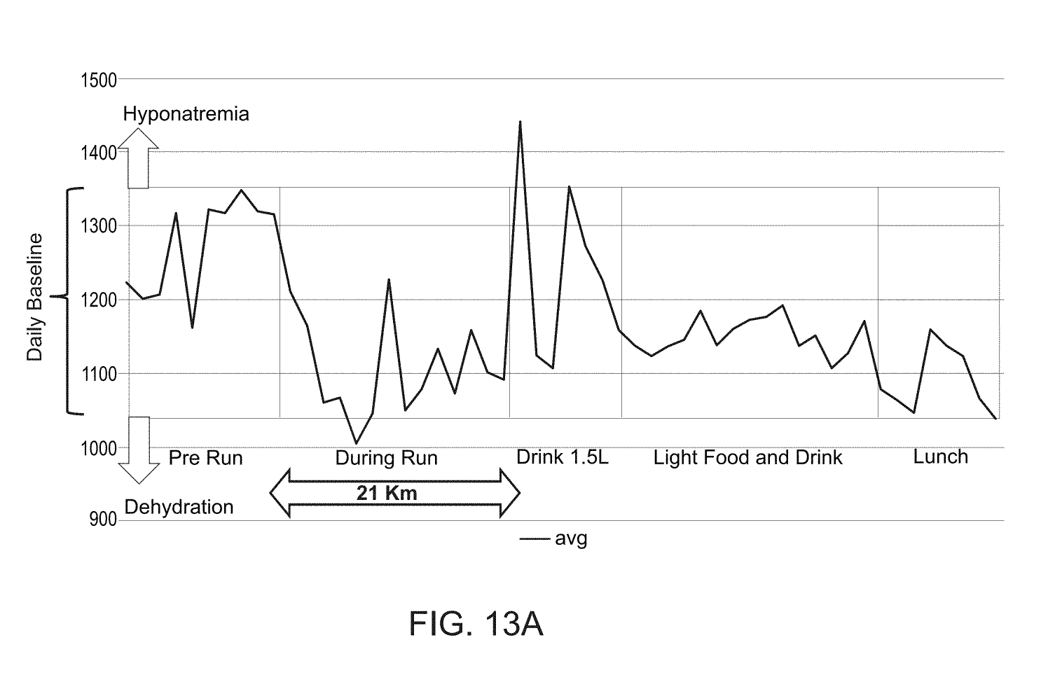

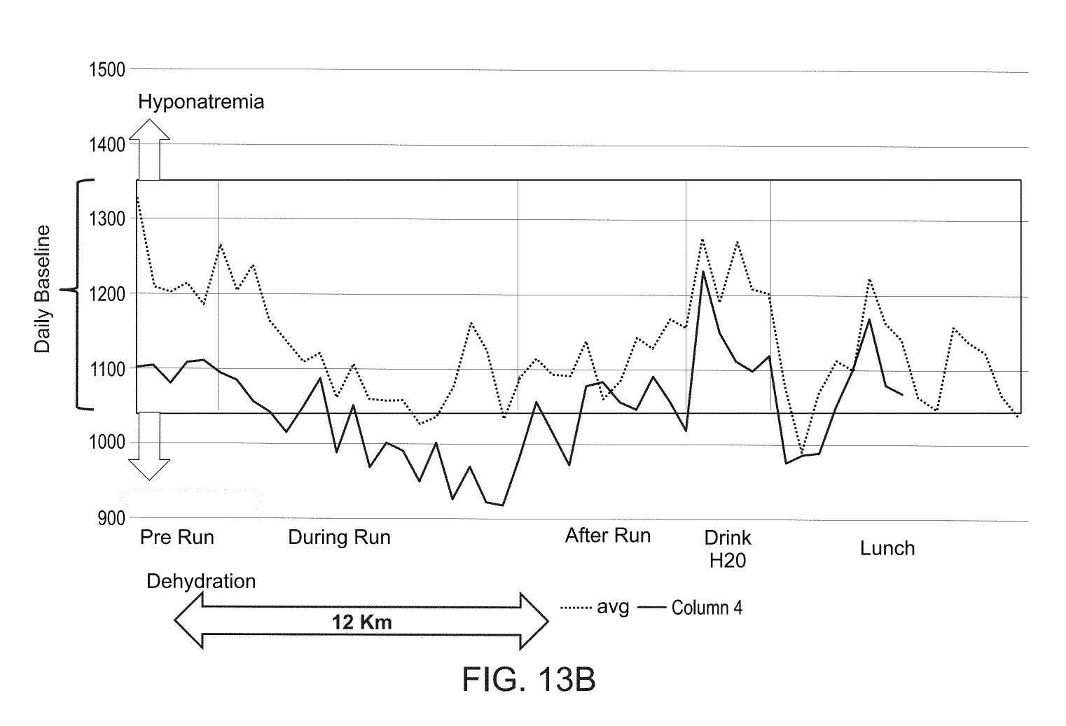

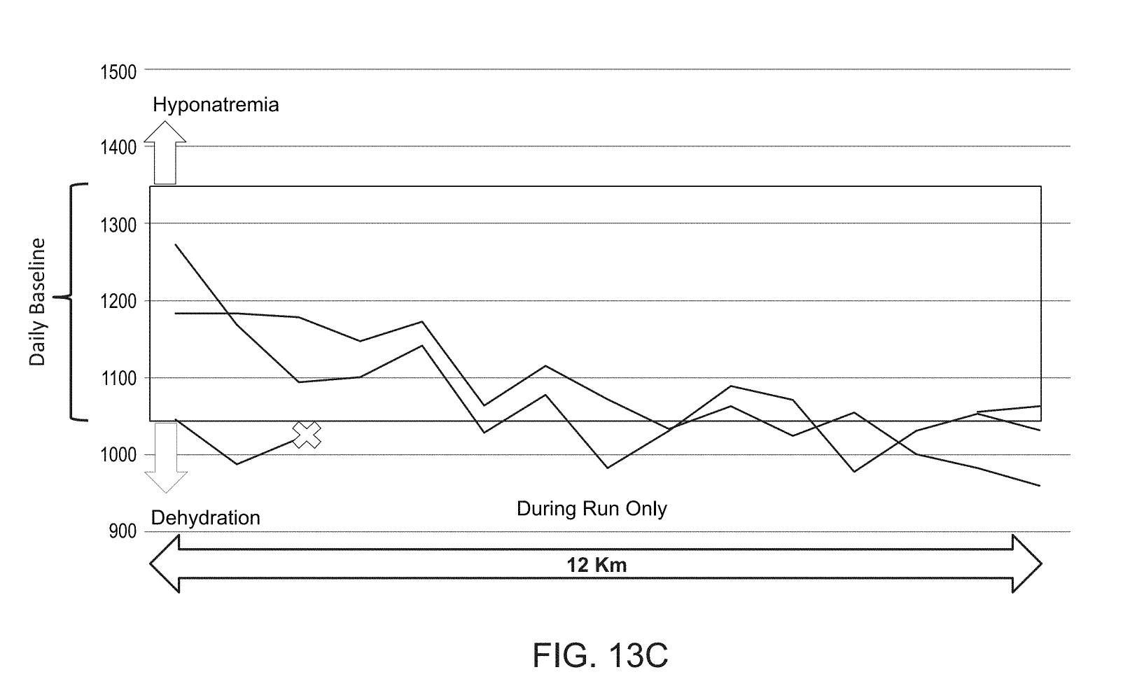

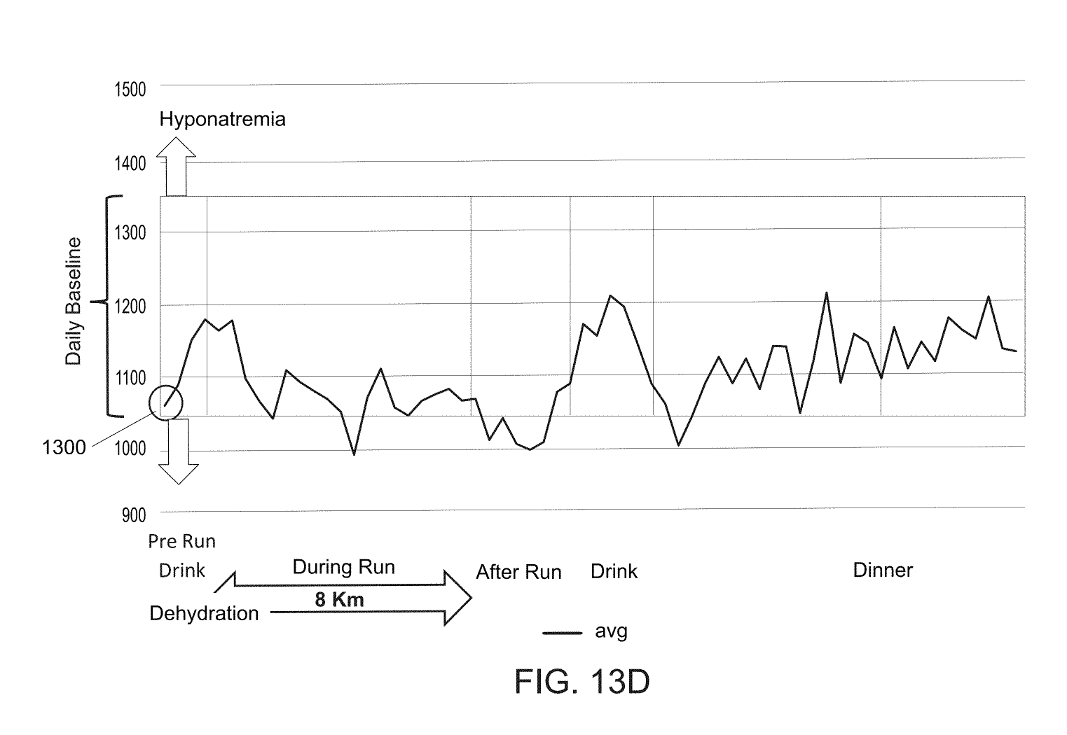

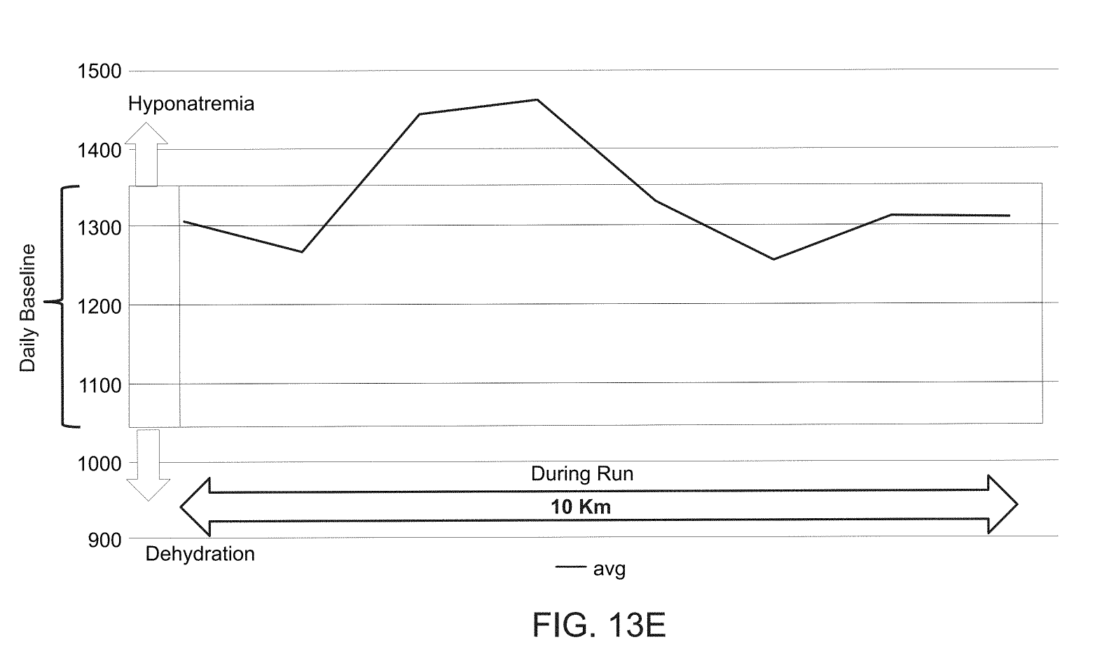

FIGS. 13A-E present results of tongue impedance measurements performed for a plurality of subjects before, during and following exercise (running), in accordance with some embodiments. In this series of experiments, tongue impedance levels were measured for subjects before, during and after long runs. Some of the measurements were obtained with respect to consumption of food and water. Subsequent measurements were performed with a time interval of between 10 to 20 minutes between them. FIG. 13A graphically presents the measured impedance values of a subject before, during and after running 21 km. As can be observed, the measurements indicated that the subject reached a dehydration state during the run, and a state of hyponatremia upon consuming a large amount of fluid after completing the run. FIG. 13B graphically presents the measured impedance values of 2 runners before, during and after running 12 km. Measurements of both runners during a part of the run and at times following the run indicated a dehydration state at some of the measured time points. FIG. 13C graphically presents the measured impedance values of 3 runners during a 12 km run, where one of the subjects discontinued the run after 1 km due to exhaustion and sickness. As can be observed, measurements acquired from the subject that discontinued the run indicated a state of dehydration. FIG. 13D graphically presents the results of a subject before, during and after running 8 km. Before the run, as indicated by the circled area 1300, the subject consumed 400 cc of water, causing a rise in the measured impedance. FIG. 13E graphically presents the results of a subject during a 10 km run. This subject was trained at running marathons. The subject consumed magnesium for several months before the run (which in some cases, if taken in a non-restricted manner may cause electrolyte imbalance, such as hyponatremia.). This subject suffered from diarrhea for several months before the run, and fainted twice during that time period. Immediately following the run, the subject consumed salt pills to assist recovery.

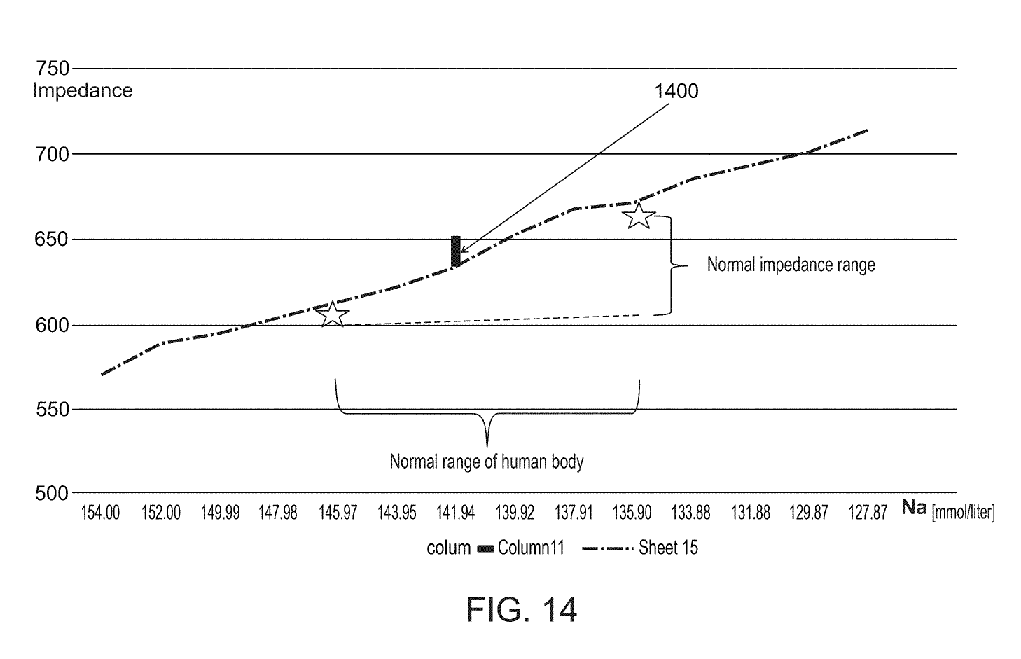

FIG. 14 presents results of an in vitro experiment performed by the inventors in which impedance was measured in a physiological solution at various sodium concentrations. The inventors used a solution in which the sodium chloride concentrations were gradually increased, and for each concentration an impedance level was measured by immersing the device in the solution. To imitate contact with tissue, the inventors modified electrode properties—the electrodes were reduced in size and relocated across the device such that a higher distance existed between electrode pairs. These changes were performed for obtaining an impedance range closer to a range which would have been measured on tongue tissue, taking into account that electrode contact with tissue is not as good as electrode contact with a solution in which it is immersed. As can be observed, the impedance levels ascended as the concentration increased.

This experiment further included adding potassium to the solution to imitate body electrolyte levels. At 1400, impedance was measured at first for a potassium concentration of 3 mmol and then for a potassium concentration of 5 mmol, in accordance with normal body potassium levels. The results obtained suggested that the change in potassium levels had only a minor effect on the measured impedance as compared to the effect caused by changing sodium levels.

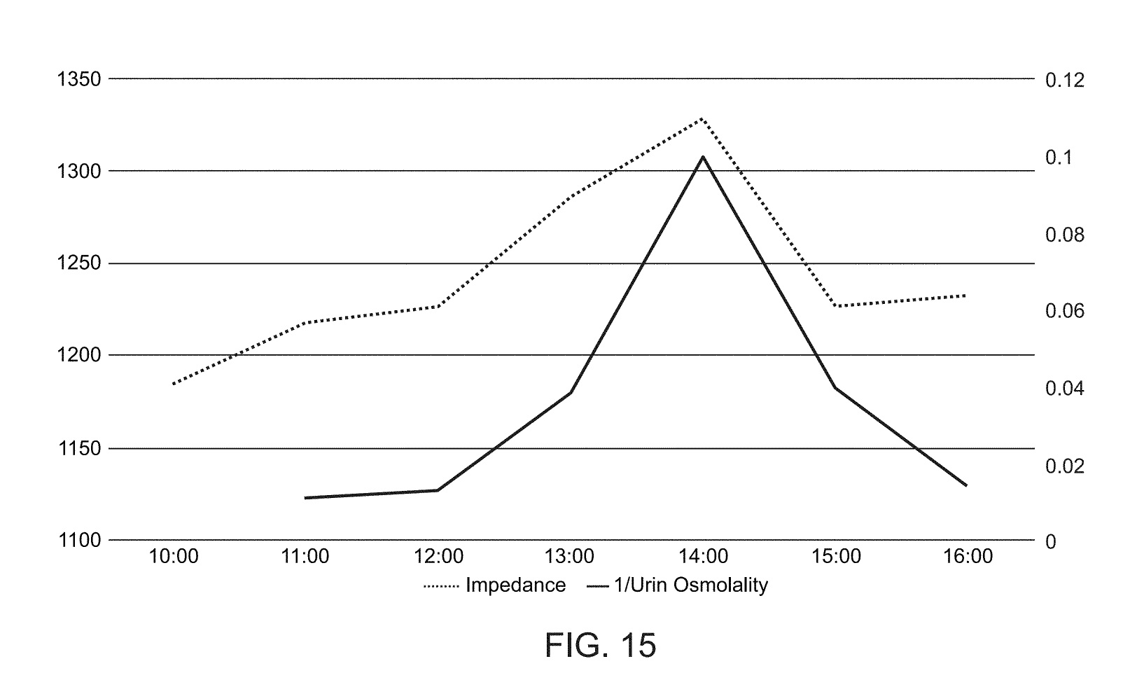

FIG. 15 presents results of a comparison between tongue impedance levels and urine osmolality in a subject, in accordance with some embodiments. A urine sample was obtained every hour from the same subject and tested using an osmolality meter. The presented comparison proves a correlation between the two measures. In some cases, a change in urine osmolality is observed only a time period after a change in impedance is detected—this may be a result of a natural physiological delay.

FIGS. 16A-B are side view and top view images of a device 1600 for measuring tongue tissue impedance, in accordance with some embodiments. In some embodiments, device 1600 comprises a handle 1602, an insert 1604 to be placed in contact with the tongue, and a positioning element 1606 (retainer) for holding the insert in place. In some embodiments, handle comprises a screen 1608 for presenting the detected results. In the exemplary device shown, an electrode configuration of the insert is for example as described hereinabove in FIG. 4B, comprising an arrangement of 8 electrodes 1610 and associated circuitry 1612.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon. Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

1. A method for measuring tongue tissue impedance of an individual, comprising:
measuring impedance of said tongue tissue; and
assessing one or both of fluid balance and an electrolyte balance of said individual according to results of said measuring.

2. The method according to claim 1, further comprising placing at least two electrodes in contact with tongue tissue of said individual.

3-4. (canceled)

5. The method according to claim 1, wherein said measuring comprises stimulating said tongue tissue using said electrodes and recording a response of said tissue to said stimulation.

6-9. (canceled)

10. The method according to claim 1, further comprising comparing impedance results of said measuring to an impedance scale, said scale defining upper and lower limits of impedance values associated with a certain condition out of a plurality of different fluid conditions and/or electrolyte conditions.

11. The method according to claim 2, wherein said placing comprises arranging said electrodes on tongue portions in which the tissue exhibits high conductance properties relative to other tongue portions.

12. The method according to claim 2, wherein said placing comprises placing at least 4 electrodes, and wherein said measuring comprises collecting impedance samples by pairing, per each time sample collected, different electrodes selected out of electrodes that are in contact with tongue tissue out of said at least four electrodes.

13. A device for measuring tongue tissue impedance as an indication of one or both of body fluid balance and an electrolyte balance of an individual, comprising:
at least two electrodes sized to fit within the mouth to be positioned in contact with tongue tissue of said individual, said electrodes disposed at a distance from each other; and
a controller configured for activating said at least two electrodes to measure impedance of said tongue tissue as an indication of one or both of body fluid balance and an electrolyte balance of said individual.

14-19. (canceled)

20. The device according to claim 13, further comprising a positioning element for holding said electrodes in the mouth, said positioning element shaped and sized to be propped against one or more anatomical structures of the mouth, internal or external.

21. The device according to claim 20, wherein said positioning element is shaped to engage the external surface of the lips.

22. The device according to claim 20, wherein said positioning element is shaped as a retainer.

23. The device according to claim 20, wherein said positioning element is shaped to center a PCB on which said electrodes are mounted relative to the tongue.

24. The device according to claim 13, further comprising a handle configured distally to said element insertable into the mouth, said handle extending externally to the mouth for holding by a user.

25-27. (canceled)

28. The device according to claim 13, wherein said element insertable into the mouth comprises a fuse that is short-circuited at the end of use.

29. The device according to claim 24, wherein said handle comprises a mechanism for deforming at least a portion of said element insertable into the mouth to disable its function when disconnected from said handle.

30-31. (canceled)

32. The device according to claim 13, wherein said device is incorporated in a mouth piece.

33-34. (canceled)

35. The device according to claim 13, further comprising an air blister configured to be positioned between said electrodes and a roof of the user's mouth, said air blister configured to force said electrodes towards the tongue tissue to increase contact between the electrodes and the tissue.

36-39. (canceled)

40. A system for assessing one or both of body fluid balance and an electrolyte balance of an individual, comprising:
a device according to claim 13;
a user interface;
a communication module; and
a memory.

41-42. (canceled)

43. The system according to claim 40, wherein said user interface configured to provide one or more of a visible indication, an audible indication, and a tactile indication to the user; and wherein said indication comprises one or both of a fluid balance and an electrolyte balance of said user.

44-47. (canceled)

48. The system according to claim 40, wherein for impedance values lower than 1040 ohm said system is configured to provide a dehydration alert and for impedance values higher than 1350 ohm said system is configured to provide a hyponatremia alert.

49. The system according to claim 40, wherein said system is configured to monitor tongue impedance levels of a user and to generate a fluid consumption recommendation accordingly.

50. The system according to claim 40, wherein said system is configured to generate a fluid and/or food consumption recommendation for a user based on a correlation between previously acquired measurements and personal physical performance levels of the user.
